(19) United States
(12) Patent Application Publication
(10) Pub. No.: US 2018/0236194 A1
(43) Pub. Date: Aug. 23, 2018

(54) TRACHEOSTOMY TUBE ASSEMBLIES AND INNER CANNULAE

(71) Applicant: SMITHS MEDICAL INTERNATIONAL LIMITED, Ashford, Kent (GB)

(72) Inventors: Timothy BATEMAN, Dymchurch (GB); Stephen James FIELD, Bridge (GB); Andrew Thomas JEFFREY, Hythe (GB); Christopher John WOOSNAM, Great Sutton (GB)

(73) Assignee: Smiths Medical International Limited, Ashford, Kent (GB)

(21) Appl. No.: 15/752,756

(22) PCT Filed: Aug. 15, 2016
(86) PCT No.: PCT/GB2016/000151

§ 371 Date: Feb. 14, 2018

(30) Foreign Application Priority Data
Aug. 29, 2015 (GB) 1515450.3

(51) Int. Cl. A61M 16/04

(52) U.S. Cl. CPC A61M 16/0465; B29C 69/02; A61M 16/0434; A61M 2207/00

(57) ABSTRACT

An inner cannula (200) for a tracheostomy tube (100) has an extruded shaft (20) with a smooth bore. A helical reinforcement element (23) is moulded onto the outside of the shaft the axial strength of which is increased by multiple groups of short bridging elements (24) extending between two adjacent turns of the reinforcement element. The bridging elements (24) are spaced around and along the shaft (20).

This invention relates to tracheostomy tube assemblies of the kind including an outer tracheostomy tube and an inner cannula removably inserted to extend along the bore of the outer tube.

Tracheostomy tube assemblies commonly include an outer tube and an inner tube or cannula that is a removable fit within the outer tube. The inner cannula can be removed and replaced periodically to ensure that the passage through the assembly does not become blocked by secretions. This avoids the need to remove the outer tube frequently.

The inner cannula presents various problems because it must be thin walled and a close fit within the outer tube so as to provide a large bore and thereby limit the resistance to flow of gas along the assembly. It must, however, also be sufficiently stiff to be inserted in the outer tube without buckling or kinking and must be readily removable, preferably with only minimal force being exerted on the tube. WO94/01156 and WO2004/101048 describe inner cannulae made of PTFE. EP1938857 describes an arrangement of tracheostomy tubes and inner cannulae where the hubs of the inner cannulae of different sizes are shaped differently so that they will only fit in the appropriate tracheostomy tube. EP2224985 describes an arrangement for attaching a hub to the shaft of an inner cannula. GB2056285 describes an inner cannula having a wall corrugated both externally and internally and a longitudinal groove or other reinforcement member traversing at least some of the corrugations. U.S. Pat. No. 4,817,598 describes a smooth-walled inner cannula having a ring-pull formation at its rear, machine end. U.S. Pat. No. 5,119,811 describes an inner cannula with a flared patient end and formed of two layers of different materials. U.S. Pat. No. 5,386,826 describes an inner cannula with an outer helical filament or layer of low friction material. U.S. Pat. No. 5,983,895 describes an inner cannula with straight sections at opposite ends joined by an intermediate curved section. U.S. Pat. No. 6,019,753 describes an inner cannula with two elongate regions of different flexibility so that the cannula has a plane of preferential bending. U.S. Pat. No. 6,019,753 describes an inner cannula having a shaft formed with slots to make it more flexible, the slots being covered by an outer thin sheath. U.S. Pat. No. 6,135,110 describes a curved inner cannula that is retained with the outer tube by means of a rotatable spring fitting. Other inner cannula arrangements are described in, for example, U.S. Pat. No. 6,024,730, WO2014/132015, WO2014/32016, WO2015/110773, WO2015/118288, WO2015/136232, WO2015/145099, WO2015/166200, GB2531902 and PCT/GB2016/000069.

It is an object of the present invention to provide an alternative tracheostomy tube assembly and inner cannula.

According to one aspect of the present invention there is provided a tracheostomy tube assembly of the above-specified kind, characterised in that the inner cannula has at least one helical element extending along the outer surface of the shaft of the cannula and a plurality of bridging elements extending between two adjacent turns of the helical element at locations along the cannula so as to limit axial compression of the cannula.

The bridging elements are preferably spaced around the circumference of the cannula and spaced along its length. The bridging elements may be arranged in two or more groups spaced around the cannula and aligned with one another at spaced locations along the cannula. The bridging elements are preferably arranged in three groups spaced around the cannula by 120° from each other. The shaft of the cannula, the helical element and the bridging elements are preferably each of a plastics material. The shaft of the cannula preferably has a smooth internal bore. The helical element and the bridging elements are preferably formed separately of the shaft of the cannula by moulding onto the outside of the shaft.

According to another aspect of the present invention there is provided an inner cannula for a tracheostomy tube assembly according to the above one aspect of the present invention.

According to a further aspect of the present invention there is provided an inner cannula for removable insertion along the bore of a tracheostomy tube, characterised in that the inner cannula has at least one helical element extending along the outer surface of the shaft of the cannula and a plurality of bridging elements extending between two adjacent turns of the helical element at locations along the cannula so as to limit axial compression of the cannula.

According to a fourth aspect of the present invention there is provided a method of making an inner cannula for a tracheostomy tube including the steps of forming a shaft of a plastics material and having a substantially smooth inner and outer surface along the major part of its length, and subsequently moulding onto the outer surface of the shaft a helical reinforcing element and a plurality of bridging elements extending between two adjacent turns of the helical element at locations along the shaft.

The shaft of the inner cannula is preferably made by extrusion.

According to a fifth aspect of the present invention there is provided an inner cannula made by a method according to the above fourth aspect of the present invention.

A tracheostomy assembly including an inner cannula and its method of manufacture, in accordance with the present invention, will now be described, by way of example, with reference to the accompanying drawings, in which:

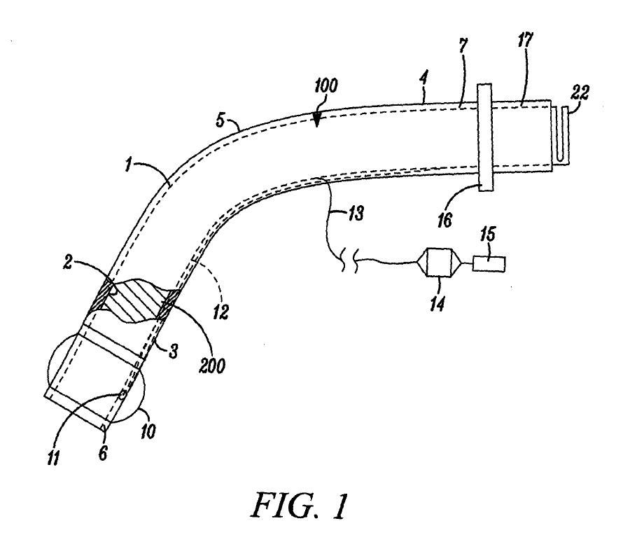

FIG. 1 is a side elevation view of the tracheostomy assembly;

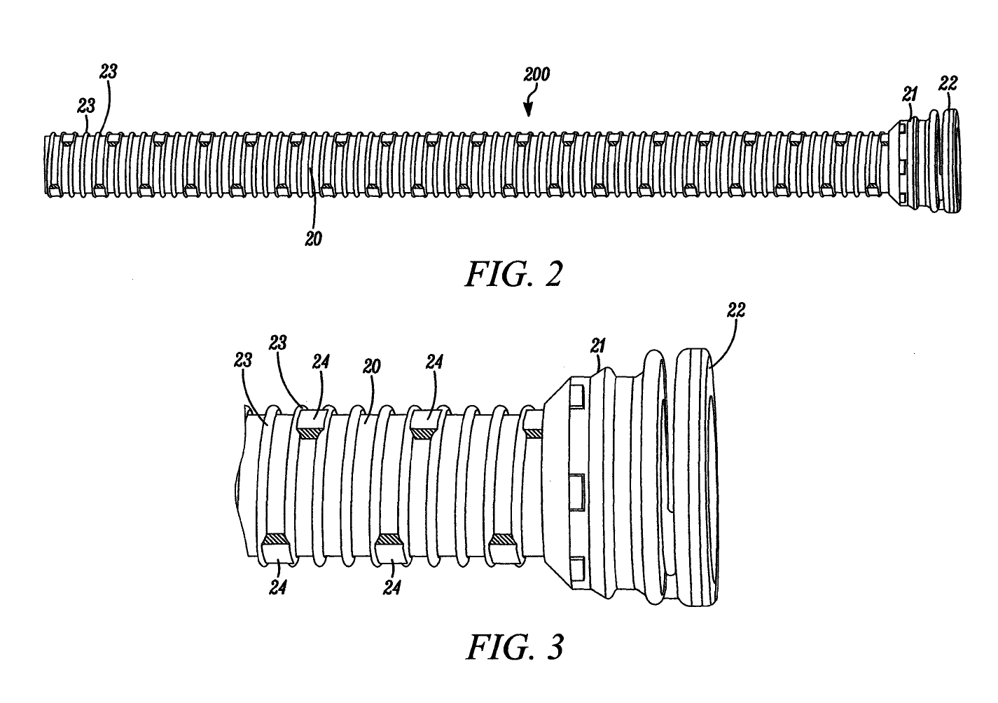

FIG. 2 is a side elevation view of the inner cannula of the assembly;

FIG. 3 is an enlarged side elevation view of the machine end of the inner cannula;

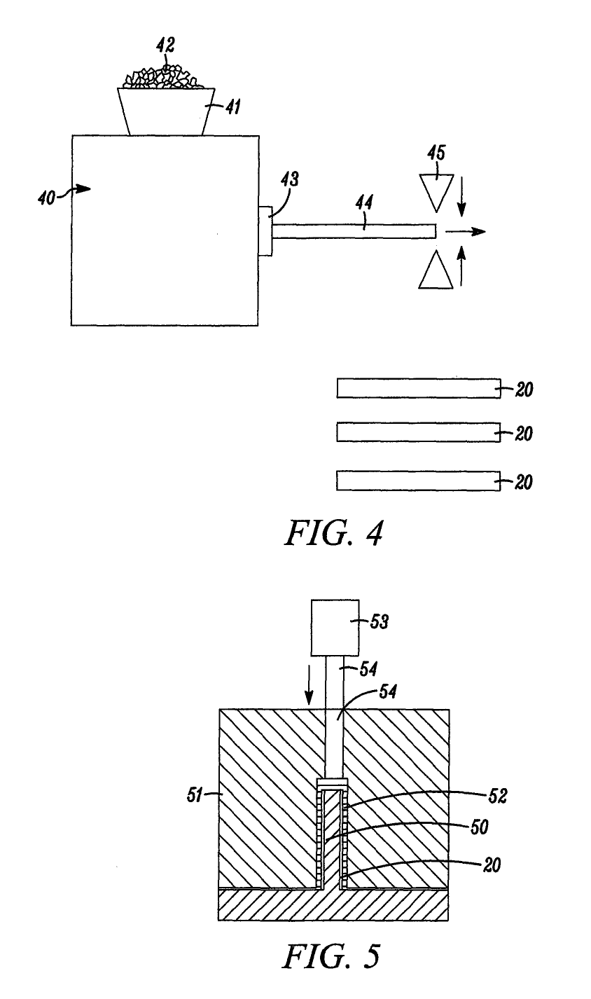

FIG. 4 illustrates an extrusion step in manufacture of the inner cannula; and

FIG. 5 illustrates a subsequent step in manufacture of the inner cannula during which a reinforcement element and bridging elements are moulded onto the outside of the inner cannula.

With reference first to FIG. 1, the tracheostomy tube assembly comprises a tracheostomy tube 100 and an inner cannula 200 inserted within the tube. The tube 100 includes a tubular shaft 1 having a bore 2 extending along its length. The tube 100 is formed with a relatively straight patient end portion 3 and a relatively straight machine end portion 4 linked by a curved intermediate portion 5 so that the patient and machine ends 6 and 7 are angled at about 100° to one another. The shaft 1 is extruded or moulded from a plastics material such as PVC or silicone. Towards its patient end 6 the tube 100 has sealing means provided by an inflatable cuff 10 embracing the shaft 1. The cuff 10 is of the high-volume/low-pressure kind so that it has a relatively floppy shape when deflated but, when inflated, it fills out at low pressure to a diameter just larger than the internal diameter of the trachea, so that it contacts the inside of the trachea with low pressure. The cuff 10 is moulded to the desired inflated shape from a plastics material such as PVC or polyurethane. The cuff 10 is attached to the shaft 1 at opposite ends over an opening 11 on the outer surface of the shaft into an inflation lumen 12 extending along the shaft within its wall thickness. The inflation lumen 12 is connected towards the rear end 7 of the tube 100 with a small-bore inflation line 13 that is terminated by an inflation indicator 14 and a valve 15. At its machine end 7 the tube 100 has a flange 16 and female connector 17 of conventional form.

The inner cannula 200 includes a shaft 20 of circular section and of a thin, stiff plastics material, such as PVC, polyurethane, polyethylene, polypropylene, PTFE or other flexible or semi-rigid plastics material. The external diameter of the shaft 20 is selected, along most of its length, to be just smaller than the inner diameter of the shaft 1 of the outer tube 100 so that the inner cannula 200 can be readily inserted and removed from the outer tube. The shaft 20 is formed straight but is highly flexible. The rear or machine end of the cannula 200 has an integral hub or machine end fitting 21 with a thicker wall than the shaft 20. The hub or machine end fitting 21 is shaped to locate and secure within the connector 17 at the machine end 7 of the tracheostomy tube 100. When the inner cannula 200 is fully inserted in the tube 100 a short part of the hub 21 projects rearwardly from the connector 17 on the tube to enable the cannula to be gripped and removed from the outer tube when necessary. The rear end of the hub 21 may have a ring-pull member 22 to facilitate removal.

As so far described, the inner cannula 200 is conventional. The cannula 200, however, differs from previous cannulae in having a helical reinforcement element 23 extending around and along the length of the outside of the shaft 20. The reinforcement element 23 is a single-start helix but could be a multiple-start helix. The element 23 is of a plastics material, which may be the same as or different from that of the shaft 20 providing the element can be securely attached to the outside of the shaft. The width of the reinforcement element 23 is preferably about 10% of the external diameter of the shaft 20 and the pitch between adjacent turns of the element is preferably about 15% of the external diameter of the shaft. The external surface of the inner cannula 200 is also provided with multiple short bridging elements 24 in the form of small blocks of plastics material with a length equal to the pitch of the helical element 23 and attached with the external surface of the shaft 20. The bridging elements 24 are discontinuous and are separate from one another. The bridging elements 24 extend between two adjacent turns of the helical reinforcement element 23 with the outer surface of the bridging elements lying level with the outer edge of the reinforcing element. It can be seen that the bridging elements 24 do not extend beyond the gap between one pair of adjacent turns of the helical element 23. There are three groups of bridging elements 24 spaced from one another around the cannula 200 by 120° with respective bridging elements in each group being aligned in three sets of elements extending longitudinally along the cannula. The helical reinforcement element 23 serves to strengthen the cannula 200 and increase its resistance to kinking, allowing the cannula to be bent and flexed through larger angles than would be possible without the reinforcement element before it kinks and buckles or collapses. The bridging elements 24 control the axial rigidity and compressibility of the cannula 200 enabling the amount of axial compression of the cannula 200 for a given axial force to be controlled by appropriate selection of the number and disposition of the bridging elements. These functions can be achieved whilst retaining a smooth bore along the inside of the inner cannula 200.

The inner cannula 200 could be made in various different ways such as by a single injection moulding process if the shaft 20 and reinforcement helix 23 and bridging elements 24 are all of the same material. Alternatively, if different materials were needed, the reinforcement and bridging elements could be overmoulded from a different material onto a preformed shaft. The machine end hub 21 could be moulded onto the end of the shaft 20, or a preformed hub could be bonded in place either before or after the reinforcement elements 23 and bridging elements 24 were formed.

FIGS. 4 and 5 show an alternative method of manufacturing the inner cannula 200. First, as shown in FIG. 4, an extruder machine 40 with a hopper 41 of thermoplastics granules 42 extrudes a continuous length of tubing 44 through its die 43. A cutter 45 cuts the tubing 44 into pre-set lengths equal to those needed for the shaft 20 of the inner cannula 200. Each shaft 20 is then placed onto a mandrel 50 of an injection moulding tool 51 (FIG. 5) within a mould cavity 52 the outer surface of which is formed with the pattern needed to produce the reinforcement helix 23 and bridging elements 24. Liquid plastics is then injected into the mould cavity 52 from a pump 53 via a flow passage 54 into the recess between the outer surface of the shaft 20 and the formations on the surface of the cavity. In this way, the reinforcement element and bridging elements can be of the same material as or of a different material from that of the shaft 20. When the moulded plastics has cooled and set the finished cannula 200 can be removed from the mould tool 51 and mandrel 50.

The present invention provides an inner cannula that can be highly flexible and yet have sufficient axial strength to enable it to be pushed into a tracheal tube, even when it is a close fit in the bore of the tracheal tube, without the risk of the cannula kinking and collapsing. This enables the inner cannula to have a relatively large internal diameter to maximise gas flow along the assembly.

1-12 (canceled)

13. A tracheostomy tube assembly including an outer tracheostomy tube and an inner cannula removably inserted to extend along the bore of the outer tube, characterised in that the inner cannula has at least one helical element extending along the outer surface of the shaft of the cannula and a plurality of bridging elements extending between two adjacent turns of the helical element at locations along the cannula so as to limit axial compression of the cannula.

14. An assembly according to claim 13, characterised in that the bridging elements are spaced around the circumference of the cannula and spaced along its length.

15. An assembly according to claim 14, characterised in that the bridging elements are arranged in two or more groups spaced around the cannula and aligned with one another at spaced locations along the cannula.

16. An assembly according to claim 15, characterised in that the bridging elements are arranged in three groups spaced around the cannula by 120° from one another.

17. An assembly according to claim 13, characterised in that the shaft of the cannula (200), the helical element and the bridging elements are each of plastics material.

18. An assembly according to claim 13, characterised in that the shaft of the cannula has a smooth internal bore.

19. An assembly according to claim 13, characterised in that the helical element and the bridging elements are formed separately of the shaft of the cannula by moulding onto the outside of the shaft.

20. An inner cannula for a tracheostomy tube assembly including an outer tracheostomy tube, the inner cannula removably inserted to extend along the bore of the outer tube, characterised in that the inner cannula has at least one helical element extending along the outer surface of the shaft of the cannula and a plurality of bridging elements extending between two adjacent turns of the helical element at locations along the cannula so as to limit axial compression of the cannula.

21. An inner cannula for removable insertion along the bore of a tracheostomy tube, characterised in that the inner cannula has at least one helical element extending along the outer surface of the shaft of the cannula and a plurality of bridging elements extending between two adjacent turns of the helical element at locations along the cannula so as to limit axial compression of the cannula.

22. A method of making an inner cannula for a tracheostomy tube including the steps of forming a shaft of a plastics material and having a substantially smooth inner and outer surface along the major part of its length, and subsequently moulding onto the outer surface of the shaft a helical reinforcing element and a plurality of bridging elements extending between two adjacent turns of the helical element at locations along the shaft.

23. A method according to claim 22, characterised in that the shaft of the inner cannula is made by extrusion.

24. An inner cannula made by a method according to claim 22.
